SEQUENCE LISTING (1 sequence)

SEQ ID NO: 1 - protein, 4 aa, Artificial Sequence
  synthetically generated peptide
AAPV

(19) United States
(12) Patent Application Publication
(10) Pub. No.: US 2017/0266182 A1
(43) Pub. Date: Sep. 21, 2017

(54) Treatment of IgG-Immune Complex-Mediated Organ Damage

(71) Applicant: The Brigham and Women's Hospital, Inc., Boston, MA (US)

(72) Inventors: Tanya Mayadas, Jamaica Plain, MA (US); Hiroshi Nishi, Brookline, MA (US)

(21) Appl. No.: 15/309,890

(22) PCT Filed: May 8, 2015
(86) PCT No.: PCT/US15/29831

§ 371 Date: Nov. 9, 2016

Related U.S. Application Data
(60) Provisional application No. 61/990,873, filed on May 9, 2014.
(60) Provisional application No. 62/048,592, filed on Sep. 10, 2014.

(51) Int. Cl. A61K 31/496; A61K 31/506

(52) U.S. Cl. CPC A61K 31/496; A61K 31/506

(57) ABSTRACT

Methods for treating IgG-immune complex-mediated organ damage, e.g., in lupus or Goodpasture syndrome, using Bcr-Abl tyrosine-kinase inhibitors, e.g., Bosutinib.

### CLAIM OF PRIORITY

This application claims the benefit of U.S. Provisional Patent Application Ser. Nos. 61/990,873, filed on May 9, 2014, and 62/048,592, filed on Sep. 10, 2014. The entire contents of the foregoing are hereby incorporated by reference.

### FEDERALLY SPONSORED RESEARCH OR DEVELOPMENT

This invention was made with Government support under Grant No. NHLBI HL065095-15 awarded by the National Institutes of Health. The Government has certain rights in the invention.

### TECHNICAL FIELD

The present invention provides methods for treating IgG-immune complex-mediated organ damage, e.g., in lupus or Goodpasture syndrome, using bosutinib.

### BACKGROUND

FcγRs have emerged as central players in autoimmune mediated inflammation and injury. Mice deficient in the common γ-chain (γ^{-/-}), required for expression and function of the murine activating FcγRs, are protected in a wide spectrum of IgG mediated inflammatory diseases including acute and progressive glomerulonephritis, autoimmune skin disease, arthritis and SLE nephritis¹⁻². The structures of human and murine activating FcγRs on neutrophils significantly differ. Mice express FcγRIII and FcγRIV that rely on the ITAM-containing γ-chain for expression and signaling while humans express a single polypeptide ITAM-containing FcγRIIA and the glycophosphotidylinositol (GPI)-linked FcγRIIIB¹. FcγRIIA has been reported to positively regulate the activity of Toll-like receptors (TLRs)³⁻⁴, integrins and cytokine receptors³, which predicts multiple roles for this receptor in disease pathogenesis. Moreover, FcγRIIA small nucleotide polymorphisms (SNPs) are associated with a variety of autoimmune diseases from SLE⁵⁻⁶ and rheumatoid arthritis⁷ to inflammatory bowel disease⁸ and Type I diabetes⁹. Thus, inhibitors of this receptor have the potential to treat a range of autoimmune diseases associated with FcγRIIA activation.

Genetically modified mice have been generated to understand the physiological functions of the uniquely human FcγRs¹⁰⁻¹². Analysis of mice generated with expression of human FcγRIIA and/or FcγRIIIB selectively on neutrophils of mice lacking their endogenous activating FcγRs (γ^{-/-} mice)¹² has revealed that human neutrophil FcγRIIA is a key mediator of tissue injury in several autoimmune disease models including lupus nephritis¹³, nephrotoxic nephritis¹² and arthritis¹⁴.

### SUMMARY

The present invention is based, at least in part, on the development of methods for treating IgG-immune complex-mediated organ damage, e.g., in lupus or Goodpasture syndrome, using Bcr-Abl tyrosine-kinase inhibitors, e.g., bosutinib.

Thus, in one aspect, the invention provides methods for treating a condition associated with IgG-immune complex-mediated organ damage in a subject. The methods include administering a therapeutically effective amount of a Bcr-Abl tyrosine-kinase inhibitor to the subject.

In another aspect, the invention provides Bcr-Abl tyrosine-kinase inhibitors for use in treating a condition associated with IgG-immune complex-mediated organ damage, or for use in the manufacture of a medicament for treating a condition associated with IgG-immune complex-mediated organ damage.

In some embodiments, the condition is lupus, e.g., lupus nephritis.

In some embodiments, the condition is Goodpasture syndrome.

In some embodiments, the organ damage is kidney, liver, pancreas, lung, heart/cardiovascular system, or joint damage; in preferred embodiments, the organ damage is kidney damage.

In some embodiments, the Bcr-Abl tyrosine-kinase inhibitor is Bosutinib, Imatinib, Dasatinib, Nilotinib, Ponatinib, INNO-406, Saracatinib (AZD0530), MK-0457 (VX-680) or MK-0457 (VX-680); in preferred embodiments, the Bcr-Abl tyrosine-kinase inhibitor is Bosutinib.

In some embodiments, the subject does not have chronic myelogenous leukemia (CML).

In some embodiments, the methods include selecting a subject for treatment on the basis that they have lupus or Goodpasture syndrome.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the invention will be apparent from the following detailed description and figures, and from the claims.

### DESCRIPTION OF DRAWINGS

FIGS. 1A-K. A high-throughput small molecule screen identified bosutinib as the most potent inhibitor of FcγRIIA signaling in neutrophils. A) Mouse bone marrow neutrophils (BMN) isolated from FcγRIIA⁺/γ^{-/-} mice were loaded on 384-well format plates coated with immune complexes (ICs). Immediately thereafter, library compounds (light grey, final concentration of 10 μM) were dispensed and one hour later fluorescence was read at Ex/Em 321/421 nm. The Src inhibitor PP2 (dark grey circles along the bottom between 1 and 2), and the diluent DMSO (black circles along the top above 5) were positive and negative controls, respectively. A profile of a subset of test compounds with a representative “hit” (arrows) is shown. B) Chemical structure of bosutinib. C) Bosutinib and PP2 dose dependence in IC induced ROS generation by mouse BMNs was assessed. n=4 independent samples. D) Effect of Bosutinib on FcgRIIA cross-linking induced ROS generation was evaluated by preincubating FcγRIIA⁺/γ^{-/-} mouse BMNs with mouse anti-human FcγRIIA, adding Bosutinib at indicated concentrations and 30 min later evaluating ROS production in real time upon addition of F(ab′)2 anti-mouse IgG in a luminol-based assay. The ROS profile of neutrophils pretreated with PP2 is shown as a positive control. Representative profile of three independent experiments is shown. E) Mouse BMNs pretreated with Bosutinib at indicated concentrations for 30 minutes were subjected to FcγRIIA cross-linking, 100 μg/ml zymosan, or 100 ng/ml phorbol myristate acetate (PMA) and ROS generation was monitored. The peak level of ROS was evaluated and normalized to DMSO vehicle control samples under each condition. n=3 independent samples. F) The release of the azurophilic granule markers neutrophil MPO and elastase was assessed after plating FcγRIIA⁺/γ^{-/-} mouse BMNs on BSA or IC (BSA/anti-BSA)-coated dishes. The data are plotted as ratio of neutrophil MPO or elastase released from 3×10⁶ neutrophils per well, divided by the total cellular MPO or elastase activity after 30 minutes of adhesion to the indicated IC-coated surface (n=2 independent samples per group; Samples were assessed in duplicates). Treatment with cytochalasin B followed by 5 μM fMLP at RT for 15 minute served as a positive control. Data are representative of three independent experiments. G) BMNs pretreated with Bosutinib, PP2 or DMSO were placed on IC-coated plates and stained with Rhodamine phalloidin. Bosutinib significantly affected cell spreading. Representative pictures of adherent neutrophils are shown. PP2 served as a positive control. H) Human peripheral blood neutrophils pretreated with the indicated concentrations of Bosutinib were subjected to FcyRIIA cross-linking, using methods described in (D) and ROS generation was evaluated. I) Granule release in immune complex stimulated human peripheral blood neutrophils was evaluated as described in (F) following vehicle or Bostutinib treatment. Fmlp/Cytochalasin B treatment served as a positive control for granule release. Data is representative of three independent experiments. J) Western blot analysis of activation (phosphorylation) of indicated proteins in FcγRIIA⁺/γ^{-/-} BMN after FcyRIIA cross linking for the times given in seconds. β-actin served as a protein loading control. K) Neutrophils from wild-type mice treated with or without Bosutinib (left panel), or neutrophils from mice deficient in all src kinases and their wild-type counterparts (WT) (right panel) were subjected to FcγRIIA cross-linking and analyzed by western blot analysis for activation of indicated proteins as in J). All data are expressed as mean±standard error mean (SEM). *p<0.05, **p<0.01.

FIGS. 2A-C. Bosutinib treatment in mice attenuates FcγR mediated reactive oxygen species generation and IC mediated Reverse Passive Arthus (RPA) reaction. A) FcγRIIA⁺/γ^{-/-} mice were given an oral administration of Bosutinib (150 mg/kg) or vehicle (2% methylcellulose/0.5% Tween80 in 100 μl distilled water) by gavage, and 4 hrs later blood was drawn and peripheral blood leukocytes were purified. Cells were pre-incubated with mouse anti-human FcγRIIA at 4° C. for 30 minutes, and real-time ROS production was recorded upon addition of F(ab′)2 anti-mouse IgG. Representative data from four independent experiments is shown. B) Mice were given an oral administration of Bosutinib or vehicle as described in A) and 30 min later the cremaster reverse passive Arthus (RPA) reaction was induced by the periscrotal delivery of anti-BSA antibody and the intravenous injection of BSA. After 4 hrs, the cremaster of mice were prepared for intravital miscropy. Representative images of the cremaster are shown (left). Adherent and transmigrated cells are indicated by white and black arrows respectively. Leukocyte adhesion (middle) and transmigration (right) were quantitated (n=3 mice per group). C) The mice were treated as described in A) and 30 min later the dorsal skin RPA reaction was induced by the subcutaneous delivery of anti-BSA antibody and an intravenous injection of BSA and Evans blue dye. The skin was harvested 4 hrs later. Representative pictures of skin (upper left) and quantitation of Evans blue dye extracted from excised skin (upper middle) are shown. Representative H&E (upper right) and esterase (lower left) stained tissue sections show skin thickness indicative of dermal edema and infiltration of neutrophils (stained dark blue in original, arrows) respectively. Neutrophil accumulation in the skin tissue was also quantitated by analysis of MPO content (normalized to total protein) (lower right). n=4 mice per group. All data are mean±SEM. *p<0.05, **p<0.01.

FIGS. 3A-I. Bosutinib attenuates nephrotoxic nephritis and human SLE sera induced glomerular injury in mice. (A-C) Analysis of nephrotoxic nephritis. FcγRIIA⁺/γ^{-/-} mice were subjected to nephrotoxic nephritis by preimmunizing mice at day-3 with Complete freund's adjuvant (CFA) and rabbit IgG followed by an i.v. injection of nephrotoxic serum at day 0. Bosutinib (150 mg/kg) or a vehicle solution was administered by gavage every 24 hrs from day 0 to 21. A) Urine albumin (normalized to creatinine) at day 7, 14, and 21 was evaluated. B) The kidneys were harvested at day 21. Representative images of renal sections stained with H&E or periodic acid-Schiff (PAS) are shown (top). Glomerular crescent formation and tubulointerstitial injury were scored (graphs below, n=8-15 mice per group). C) Neutrophil accumulation in the kidney glomeruli at day 10 was evaluated on tissue sections stained with the neutrophil marker antibody NIMP-R14. D) Isolated FcγRIIA⁺/γ^{-/-} neutrophils were labeled with Cell Tracker Green CMFDA or Cell Tracker Blue CMF2HC, washed, and then incubated with Bosutinib or DMSO vehicle respectively. FcγRIIA⁺/γ^{-/-} recipient mice were injected with anti-GBM sera (+) or saline (−) and 30 mins later the Vehicle and Bosutinib treated neutrophils were injected intravenously. The mice were prepared for two-photon intravital microscopy of the glomeruli. Representative images of blue (Veh treated) and green (Bos treated) labelled neutrophils tracked over time are shown. Cumulative frequency and bar graph of the dwell time of these neutrophils in the glomeruli are given. (E-I) Analysis of lupus nephritis following the passive transfer of human SLE sera. Mice that express FcγRIIA and lack γ-chain and the integrin Mac-1 (FcγRIIA⁺/γ^{-/-} /Mac-1^{-/-}) were preimmunized with CFA at day-3, received two i.v. injections of human SLE sera on days 0 and 2 and were treated with Bosutinib or a vehicle solution by gavage every 24 hrs from day 0 to 14. E) Urine albumin (normalized to creatinine) at day 0, 7, 10, and 14 was evaluated (n=17 mice per group). F) Representative images of renal sections (day 14) stained with H&E or PAS are shown. Tissue sections were scored based on endocapillary proliferation, leukocyte infiltration and crescents and the individual scores for each parameter were added to give a total score (n=11-12 mice per group). G) Glomerular neutrophil accumulation at day 10 and 14 were evaluated as described in (C). H) Urine collected at day 14 from indicated mice were analyzed for the presence of Neutrophil Gelatinase Associated Lipocalin (NGAL). I) Human SLE sera induced arthritis was assessed every other day and given a clinical score based on redness or swelling of the entire paw (middle graph) Ankle thickness of the hind limbs (right graph) was evaluated using calipers. Representative images of lower limbs are shown (left). All data are expressed as mean±SEM. *p<0.05, **p<0.01.

FIGS. 4A-E. Abl1 is required for FcγRIIA mediated neutrophil activation. A) After pretreatment of FcγRIIA⁺/γ^{-/-} mouse BMNs with bosutinib, imatinib, or nilotinib at 100 nM, FcγRIIA cross linking reaction was induced. Data are representative of three independent experiments. The more significant effect of Bosutinib versus other Bcr-Abl inhibitors may reflect the reported ability of Bosutinib to also inhibit Src kinases, which are known downstream targets of FcγRs. B) Western blot analysis of phosphorylation (p-) of Crkl, an adaptor protein that is known to be a downstream substrate of activated Abl, in FcγRIIA⁺/γ^{-/-} BMN after FcγRIIA cross linking for times given in seconds. β-actin served as a loading control. C) Human neutrophil like HL-60 cells differentiated with 100 mM N,N-Dimethylformamide (DMF) were stably transduced with lentiviral constructs expressing two different Abl1 shRNA (Abl1 #1 and #2) sequences, and Abl1 silencing in cells was assessed by comparing expression levels of Abl1 mRNA (relative to Gapdh, a house keeping gene) and protein by quantitative real time PCR and western blot analysis, respectively. D) FcγRIIA cross linking reaction was induced in DMF differentiated HL-60 cells silenced with shRNA to Abl1 (Abl1 #1, Abl1 #2) or control shRNA and real-time ROS production was recorded (top panel). A profile of FcyRIIA cross-linking induced ROS in differentiated HL-60 cells treated with DMSO (−) or Bosutinib is shown for comparison (bottom panel). E) Differentiated HL-60 cells were placed on plates coated with IC for 20 minutes. Representative pictures of culture cells stained with phalloidin adherent to immobilized IC are shown. The area of a single spread cell was calculated by averaging the values of 20 cells per condition. The average number of adherent cells on IC was also quantified. Data are representative of three independent experiments. Data is representative of three independent experiments. **p<0.01.

### DETAILED DESCRIPTION

As described herein, the present inventors developed and performed a high-throughput chemical screen of ≈8,500 compounds that included FDA approved drugs to identify inhibitors of IgG mediated activation of primary murine neutrophils expressing human FcγRIIA and validated the results in human neutrophils. This was followed by analysis of the lead compound, Bosutinib, in FcγRIIA dependent mouse models of IC induced tissue injury¹²⁻¹³. Bosutinib, currently used for the treatment of Philadelphia chromosome positive chronic myelogenous leukemia, targets the oncogenic Bcr-Abl1 fusion protein¹⁵. Bosutinib significantly inhibited the Reverse Passive Arthus (RPA) reaction, a prototypical model of tissue injury induced by soluble ICs. Moreover, it inhibited nephrotoxic nephritis, a potential surrogate of lupus nephritis¹⁶, as well as renal injury and arthritis in a recently developed mouse model of lupus nephritis induced by the passive transfer of human SLE serum¹³. FcγRIIA cross-linking resulted in activation of Abl1 and silencing of this kinase prevented FcγRIIA induced respiratory burst suggesting that c-Abl regulates FcγRIIA mediated signal transduction linked to neutrophil cytotoxicity.

Systemic Lupus Erythematosus is a chronic, multi-organ disease with significant unmet clinical needs¹⁷⁻¹⁸. Lupus nephritis remains the leading cause of morbidity and mortality and current treatment regimens rely primarily on corticosteroids, which have significant adverse effects including organ injury¹⁹. The combination of a novel high-throughput screen and a humanized model of lupus amenable for drug screening has resulted in the identification of a drug Bostuinib, used in cancer²⁰, that may be repurposed for the treatment of lupus nephritis and arthritis, and the identification of c-Abl as a new signaling partner downstream of FcγRIIA.

Bosutinib

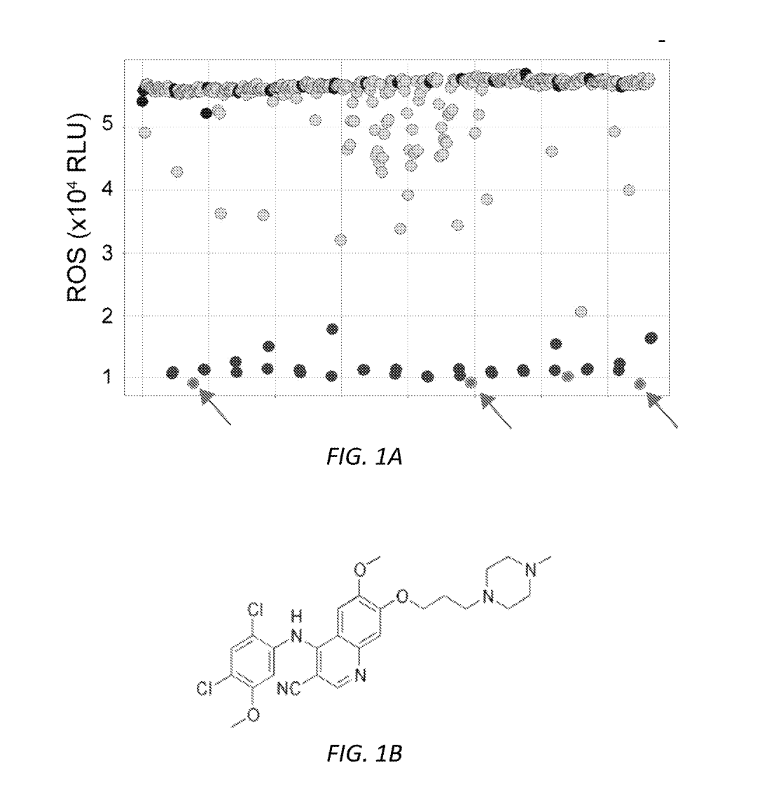

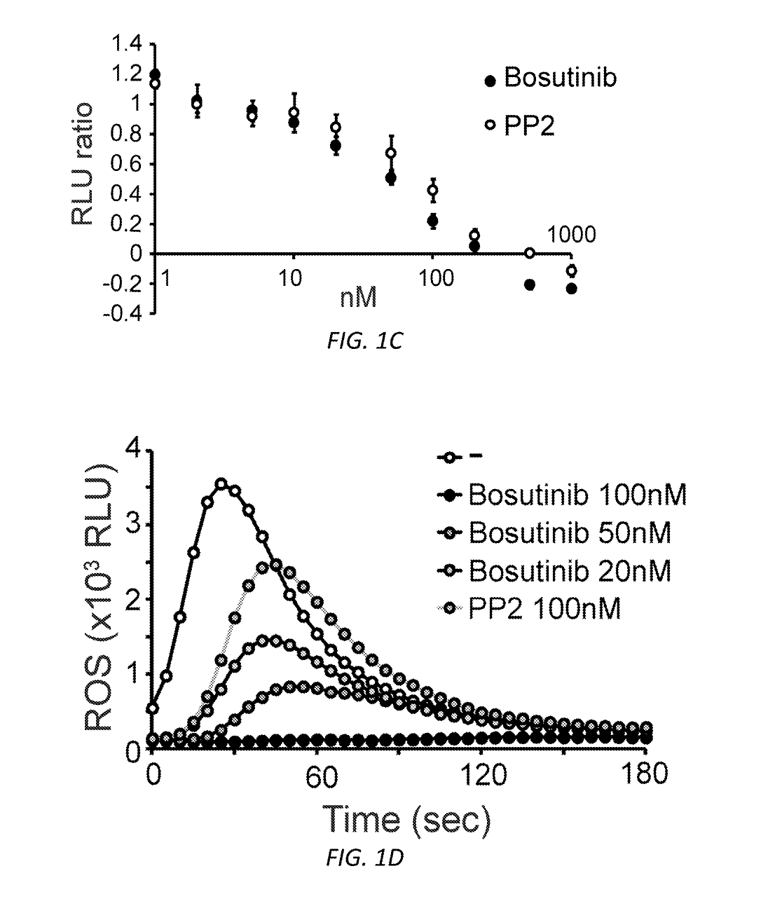

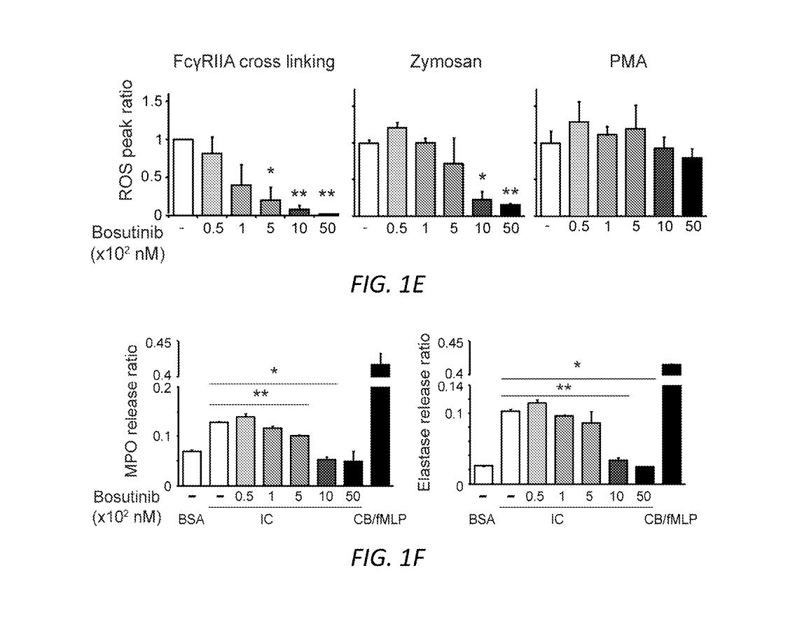

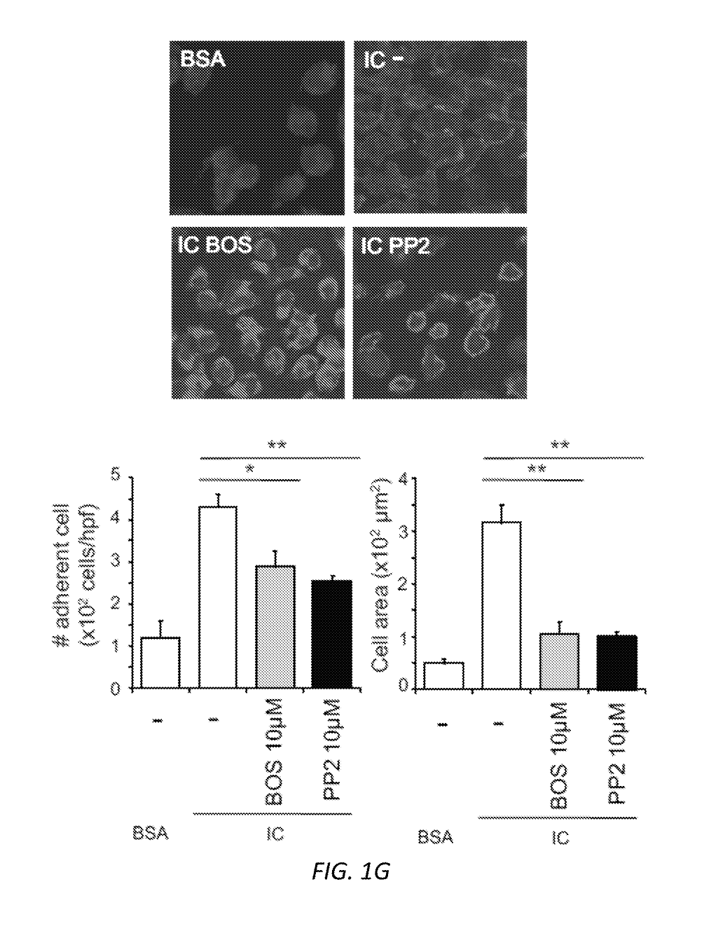

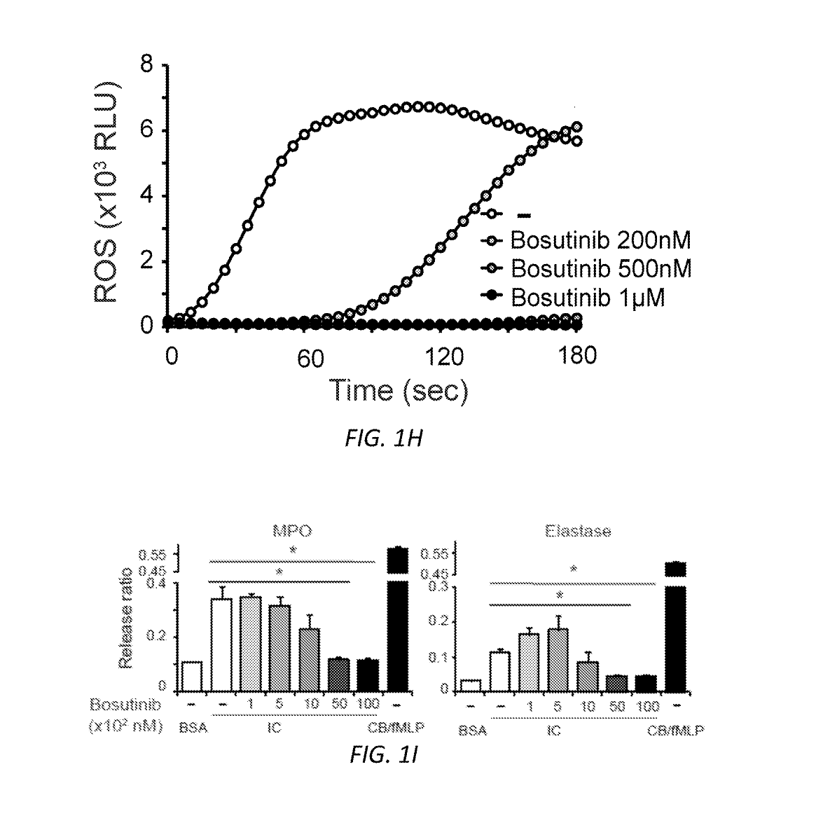

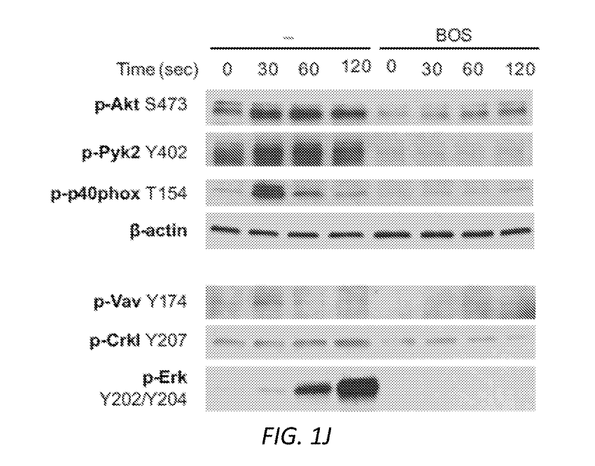

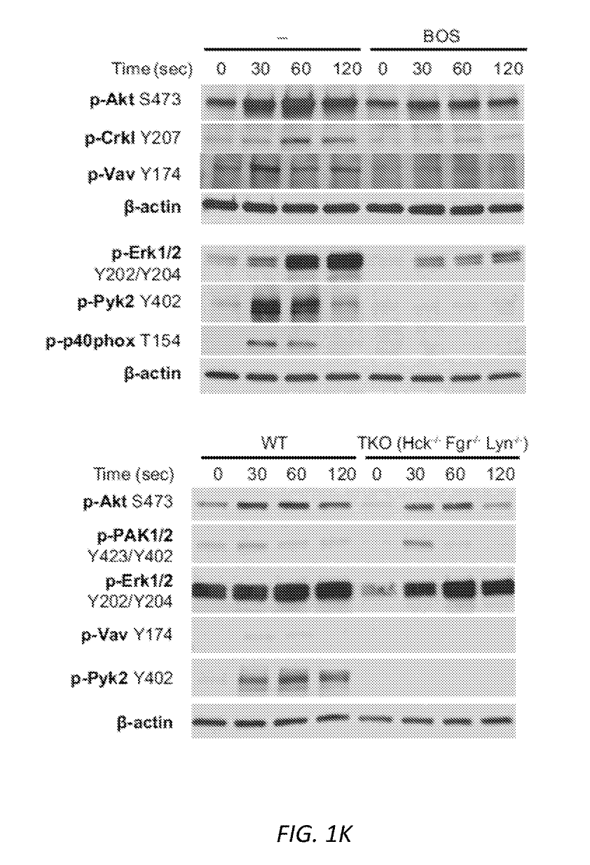

Bosutinib, a synthetic quinolone derivative, is a dual Src/Abl kinase inhibitor. The structure of bosutinib is shown in FIG. 1B. Bosutinib is sold commercially in tablet form under the brand name BOSULIF (Pfizer); the tablets include the following inactive ingredients: microcrystalline cellulose, croscarmellose sodium, poloxamer, povidone, magnesium stearate, polyvinyl alcohol, titanium dioxide, polyethylene glycol, talc, and iron oxide yellow (for 100 mg tablet) and iron oxide red (for 500 mg tablet).

Methods of Treatment

The methods described herein include methods for the treatment of disorders associated with IgG-immune complex-mediated organ damage, e.g., kidney, liver, pancreas, lung, heart/cardiovascular system, or joint damage¹. In some embodiments, the disorder is kidney damage or lupus-induced arthritis. Generally, the methods include administering a therapeutically effective amount of bosutinib to a subject who is in need of, or who has been determined to be in need of, such treatment. In some embodiments, other inhibitors are used, e.g., Imatinib, Dasatinib, Nilotinib, Ponatinib, INNO-406, AZD0530, MK-0457 (VX-680) or MK-0457 (VX-680).

As used in this context, to “treat” means to reduce the risk of developing, delay the onset or progression of, or ameliorate at least one symptom of, the disorder associated with IgG-immune complex-mediated organ damage. Often, IgG-immune complex-mediated inflammation associated with significant neutrophil influx results in organ damage, e.g., kidney, liver, pancreas, lung, heart/cardiovascular system, or joint damage. Administration of a therapeutically effective amount of a compound described herein for the treatment of a condition associated with IgG-immune complex-mediated inflammation will result in a decrease in organ damage. For example, in the case of lupus nephritis a treatment can result in a reduction in kidney damage and a return or approach to normalized renal function or, at least, in preventing the progression of loss of renal function.

Lupus

In some embodiments, the disorder is lupus, e.g., lupus nephritis. Subjects with systemic lupus erythematosus (SLE) typically have some level of nephritis even in the absence of clinical symptoms. The present methods can be used in subjects who have lupus (e.g., a confirmed diagnosis of lupus, e.g., made by a healthcare professional), but no symptoms of nephritis, or who have lupus plus one or more symptoms of lupus nephritis, laboratory tests indicating the presence of lupus nephritis, or a confirmed diagnosis of lupus nephritis, e.g., made by a healthcare professional.

Symptoms and signs of lupus may include those of active lupus (malar and other skin rashes, arthritis, pleuritis, pericarditis) and active nephritis (hypertension, peripheral edema).

SLE is diagnosed by health professionals, especially rheumatologists or nephrologists, based on the criteria established by the American College of Rheumatology (ACR) in 1996 or a revised one by the Systemic Lupus International Collaborating Clinics (SLICC) group in 2012. Lupus nephritis is characterized by findings of active glomerulonephritis and active lupus. The glomerulonephritis diagnosis is based on an active urine sediment (classes III and IV), i.e. hematuria with dysmorphic red cells, cellular casts and proteinuria, or in the case of class V just proteinuria. A renal biopsy is needed to characterize the type of renal involvement. Active lupus is diagnosed by evidence of dsDNA antibodies in the blood, low complement levels (C3 and C4) and oftentimes leukopenia and thrombocytopenia. The patients can present with evidence of renal functional compromise, i.e. increase in blood levels of BUN and creatinine but this is not necessary for the diagnosis.

Goodpasture Syndrome

In some embodiments, the disorder is Goodpasture syndrome, characterized by pulmonary hemorrhage (as seen in the images below) and acute or rapidly progressing glomerulonephritis and the presence of circulating anti-glomerular basement membrane (anti-GBM) antibodies; thus it is also referred to as anti-GBM disease.

Symptoms can include malaise, chills and fever, and/or arthralgia; hemoptysis, cough, dyspnea, chest pain and shortness of breath when the lungs are affected. Symptoms associated with renal disease include hematuria, edema, high blood pressure and eventually uremia. Massive pulmonary hemorrhage leading to respiratory failure may occur. A diagnosis can be confirmed by detecting anti-GBM antibodies, e.g., using radioimmunoassay, enzyme-linked immunosorbent assay (ELISA), or Western blotting. The present methods can be used in subjects who have anti-GBM disease (e.g., a confirmed diagnosis of anti-GBM disease, e.g., made by a healthcare professional), but no symptoms of glomerulonephritis, or who have anti-GBM disease plus one or more symptoms of glomerulonephritis, laboratory tests indicating the presence of glomerulonephritis, or a confirmed diagnosis of glomerulonephritis, e.g., made by a healthcare professional.

Pharmaceutical Compositions and Methods of Administration

The methods described herein include the use of pharmaceutical compositions comprising bosutinib as an active ingredient. In some embodiments, other inhibitors are used as an active ingredient, e.g., Imatinib, Dasatinib, Nilotinib, Ponatinib, INNO-406, Saracatinib (AZD0530), MK-0457 (VX-680) or MK-0457 (VX-680). Pharmaceutical compositions typically include a pharmaceutically acceptable carrier. As used herein the language “pharmaceutically acceptable carrier” includes saline, solvents, dispersion media, coatings, antibacterial and antifungal agents, isotonic and absorption delaying agents, and the like, compatible with pharmaceutical administration. Supplementary active compounds can also be incorporated into the compositions, e.g., immunosuppressive agents such as hydroxychloroquine, cyclophosphamide, azathioprine, or mycophenolate mofetil; steroids such as corticosteroids, e.g., methylprednisolone or prednisone; angiotensin-converting enzyme inhibitors (ACEIs); and angiotensin II receptor blockers (ARBs).

Pharmaceutical compositions are typically formulated to be compatible with its intended route of administration. Examples of routes of administration include parenteral, e.g., intravenous, intradermal, subcutaneous, oral, transdermal (topical), transmucosal, and rectal administration.

Methods of formulating suitable pharmaceutical compositions are known in the art, see, e.g., Remington: The Science and Practice of Pharmacy, 21st ed., 2005; and the books in the series Drugs and the Pharmaceutical Sciences: a Series of Textbooks and Monographs (Dekker, N.Y.). For example, solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use can include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition must be sterile and should be fluid to the extent that easy syringability exists. It should be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyetheylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as mannitol, sorbitol, sodium chloride in the composition. Prolonged absorption of the injectable compositions can be brought about by including in the composition an agent that delays absorption, for example, aluminum monostearate and gelatin.

Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle, which contains a basic dispersion medium and the required other ingredients from those enumerated above. In the case of sterile powders for the preparation of sterile injectable solutions, the preferred methods of preparation are vacuum drying and freeze-drying, which yield a powder of the active ingredient plus any additional desired ingredient from a previously sterile-filtered solution thereof

Oral compositions generally include an inert diluent or an edible carrier. For the purpose of oral therapeutic administration, the active compound can be incorporated with excipients and used in the form of tablets, troches, or capsules, e.g., gelatin capsules. Oral compositions can also be prepared using a fluid carrier for use as a mouthwash. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds can be delivered in the form of an aerosol spray from a pressured container or dispenser that contains a suitable propellant, e.g., a gas such as carbon dioxide, or a nebulizer. Such methods include those described in U.S. Pat. No. 6,468,798.

Systemic administration of a therapeutic compound as described herein can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The pharmaceutical compositions can also be prepared in the form of suppositories (e.g., with conventional suppository bases such as cocoa butter and other glycerides) or retention enemas for rectal delivery.

In one embodiment, the therapeutic compounds are prepared with carriers that will protect the therapeutic compounds against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Such formulations can be prepared using standard techniques, or obtained commercially, e.g., from Alza Corporation and Nova Pharmaceuticals, Inc. Liposomal suspensions (including liposomes targeted to selected cells with monoclonal antibodies to cellular antigens) can also be used as pharmaceutically acceptable carriers. These can be prepared according to methods known to those skilled in the art, for example, as described in U.S. Pat. No. 4,522,811.

The pharmaceutical compositions can be included in a container, pack, or dispenser together with instructions for administration.

Dosage

An “effective amount” is an amount sufficient to effect beneficial or desired results. For example, a therapeutic amount is one that achieves the desired therapeutic effect. This amount can be the same or different from a prophylactically effective amount, which is an amount necessary to prevent onset of disease or disease symptoms. An effective amount can be administered in one or more administrations, applications or dosages. A therapeutically effective amount of a therapeutic compound (i.e., an effective dosage) depends on the therapeutic compounds selected. The compositions can be administered one from one or more times per day to one or more times per week; including once every other day. The skilled artisan will appreciate that certain factors may influence the dosage and timing required to effectively treat a subject, including but not limited to the severity of the disease or disorder, previous treatments, the general health and/or age of the subject, and other diseases present. Moreover, treatment of a subject with a therapeutically effective amount of the therapeutic compounds described herein can include a single treatment or a series of treatments.

Dosage, toxicity and therapeutic efficacy of the therapeutic compounds can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Compounds that exhibit high therapeutic indices are preferred. While compounds that exhibit toxic side effects may be used, care should be taken to design a delivery system that targets such compounds to the site of affected tissue in order to minimize potential damage to uninfected cells and, thereby, reduce side effects.

The data obtained from cell culture assays and animal studies can be used in formulating a range of dosage for use in humans. The dosage of such compounds lies preferably within a range of circulating concentrations that include the ED50 with little or no toxicity. The dosage may vary within this range depending upon the dosage form employed and the route of administration utilized. For any compound used in the method of the invention, the therapeutically effective dose can be estimated initially from cell culture assays. A dose may be formulated in animal models to achieve a circulating plasma concentration range that includes the IC50 (i.e., the concentration of the test compound which achieves a half-maximal inhibition of symptoms) as determined in cell culture. Such information can be used to more accurately determine useful doses in humans. Levels in plasma may be measured, for example, by high performance liquid chromatography.

Bosutinib has regulatory approval in the United States for the treatment of certain forms of Chronic Myelogenous Leukemia; for those indications, typical dosing is 500 mg orally once daily with food. In the present method, an exemplary dose may be, e.g., 150, 200, 250, or 300 mg per day, e.g., taken orally.

### EXAMPLES

The invention is further described in the following examples, which do not limit the scope of the invention described in the claims.

### Materials and Methods

The following materials and methods were used in the Examples set forth herein.

Mice

FcγRIIA-expressing, g-chain-deficient (FcγRIIA⁺/γ^{-/-}) mice and FcγRIIA-expressing, g-chain-deficient, and Mac1-deficient (FcγRIIA⁺/γ^{-/-}/Mac1-) mice were generated as previously described^{12,13}. All mice were maintained in a virus- and antibody-free facility. Mice used for each experiment were 8-10 weeks of age and sex matched.

High Throughput Small Molecule Screening

Screen of 8,483 compounds was done in collaboration with the Institute of Chemistry and Cell Biology Screening facility at Harvard Medical School (ICCB/Longwood). Each compound was dissolved in DMSO at a concentration of 10 μM. For the primary screen, wells were coated with immune complexes as follows: 0.1 mg/mL poly-L-lysine (Sigma) followed by 2.5% glutaraldehyde were dispensed onto 384-well format black plate (Corning), and the plate was washed once with DDW and twice with PBS, incubated with 100 μL 1 mg/ml bovine serum albumin (BSA) followed by 100 μL 40 μg/mL rabbit anti-BSA antibody to generate in situ immune complexes (IC). Mature murine neutrophils isolated from the bone marrow of FcγRIIA+/γ^{-/-} mice as previously described, were suspended in 100 uM homovanillic acid/5 U/ml peroxidase IV (Sigma)/1 mM EDTA/HBSS (Thermo) assay solution (pH 7.5) and 3×10⁴ cells in 30 μL were loaded per well into a 384-well format plate using a Wellmate automated dispenser (Thermo Scientific). Immediately after dispensing cells, ICCB library compounds dissolved in 33 nL DMSO were dispensed into wells using a Compound Transfer Robot (Epson). The plates were placed in a humidified incubator at 37 C for 60 mins and then fluorescence levels were read out at Ex/Em 321/421 nm with Envision2 (PerkinElmer). PP2 (Sigma), a Src inhibitor known to down-regulate FcγRIIA signal, and DMSO were used as positive and negative control, respectively. All the experiments were performed in duplicate. For the secondary screen, the 30 most potent compounds were retested using serial dilutions (10 uM, 3.3 uM, and 1 uM) of the compound.

Human Neutrophils

Human polymorphonuclear neutrophils (PMNs; >95% pure) were isolated from whole blood drawn from healthy volunteers as previously described (Alcaide et al., Am J Physiol Cell Physiol, 303: C385-395, 2012), kept at 8° C. and used immediately. Blood was drawn and handled according to protocols for protection of human subjects approved by the Brigham and Women's Hospital Institutional Review Board, and all volunteer subjects gave written informed consent.

In Vitro Neutrophil Assays

FcγR Crosslinking-Induced Generation of ROS

10⁶ BMNs were suspended in PBS without Ca²⁺/Mg²⁺, and then incubated with 10 μg/ml mouse anti-hFcγRIIa (clone IV.3; StemCell Technologies) on ice for 30 minutes. After washing in PBS, cells were incubated with or without 500 ng/ml mGM-CSF (BioLegend) for 30 minutes. After pretreated with Bosutinib at indicated concentrations or DMSO at 37 C for 30 minutes, luminol (50 μM) in PBS with Ca²⁺/Mg²⁺ was added, followed immediately by the addition of goat anti-mouse F(ab′)2 (36 μg/ml; Jackson ImmunoResearch). ROS generation (expressed in relative light units) was continuously monitored over time using a 6-channel bioluminat LB-953 luminometer (Berthold). For human PMNs, 5×10⁵ cells were analyzed without GM-CSF pretreatment.

Zymosan or PMA Induced ROS Generation

After pretreatment with Bosutinib at indicated concentrations for 30 minutes, mouse BMNs were stimulated with 100 μg/ml zymosan (Sigma) or 100 ng/ml phorbol myristate acetate (PMA, Sigma) and ROS generation was monitored. The peak level of ROS for Bosutinib treated samples was evaluated and normalized to DMSO control (set at 1).

Degranulation Assay

3×10⁶ mouse BMNs or 1×10⁶ human PMNs were added to 24-well plates coated with immobilized BSA/anti-BSA IC and incubated at 37 C for 30 min. After centrifugation at 300 g at 4 C, 100 ul supernatant was taken for analysis. The cell pellets were lysed with 0.1% 500 ul Triton-X, centrifuged at 300 g at 4 C, and 100 ul of the sample was analyzed. For the myeloperoxidase (MPO) release assay, the substrate 3,3′,5,5′-tetramethyl-benzidine (TMB, Thermo) was added to the supernatant or cell lysate followed by a stop solution and the optical absorbance at 450 nm was measured. For neutrophil elastase, 20 uM MeO-Suc-Ala-Ala-Pro-Val-AMC (Cayman) was added and incubated at 37° C. for 24 hrs and the fluorescence intensity was read at Ex/Em 355/460 nm. The amount of MPO and elastase released from neutrophils was expressed as a ratio of the total enzyme. Cell samples treated with 5 μg/ml of cytochalasin B at 37° C. for 5 min followed by 5 μM fMLP at RT for 15 minute served as a positive control.

Adhesion and Spreading Assay

Experimental details for adhesion assay and F-actin staining were previously described¹². For adhesion analysis, adherent cell numbers were quantitated in three independent fields at magnification 200×, and the average cell number per field was determined. Neutrophils were plated on glass coverslips coated with ICs formed by BSA and polyclonal rabbit anti-BSA antibody or BSA alone in 24-well plates. For spreading analysis, the area of a single spread cell was calculated with Image-J and the values of 20 cells per condition were averaged in each condition.

Western Blot Analysis

After incubation with 10 μg/ml mouse anti-hFcγRIIa followed by mGM-CSF, 10⁵ mouse BMNs or differentiated HL60 cells suspended in PBS with Ca⁺⁺/Mg⁺⁺ were treated with 100 nM Bosutinib or DMSO for 30 min at 37 C. Cells were then treated with 1 μM diisopropyl fluorophosphates at 4 C for 10 min, lysed with 100 μL 2× Laemmli sample lysis buffer (Bio-Rad) with 2-mercaptoethanol (β-mercaptoethanol), boiled, homogenized, and run on a SDS-PAGE gel. The following Abs were used for analysis: rabbit anti-p-Abl1, rabbit anti-p-Crkl, rabbit anti-p-p40phox, rabbit anti-p-Akt, rabbit anti-p-Pyk2, and rabbit anti-p-p40phox (Cell Signaling Technology); rabbit rabbit anti-p-Vav (Santa Cruz Biotechnology); and β-actin (Sigma).

Intravital Microscopy Evaluation of the Reverse Passive Arthus (RPA) Reaction in the Cremaster Muscle

FcγRIIA+/γ^{-/-} mice were given an oral administration (gavage) of Bosutinib (150 mg/kg, 100 ul) or vehicle (2% methylcellulose /0.5% Tween80 in 100 μl distilled water) by gavage. After 30 minutes, rabbit IgG anti-BSA Ab (200 μg/300 μl) was injected periscrotally, followed by an i.v. injection of BSA (300 μg/100 μl). After 3 h, leukocyte recruitment in the cremaster of anesthetized mice was evaluated by intravital microscopy, as previously described (Tsuboi et al., Immunity, 28: 833-846, 2008). Four venules per mouse were analyzed. Leukocyte rolling velocities were measured by tracking single leukocytes (10/venule) over several frames and calculating the distance moved per unit time (μm/s). Adherent leukocytes were defined as cells remaining stationary for 30 s and were expressed as the number of cells/mm² venule.

Cutaneous RPA Reaction

Mice were given an oral administration of Bosutinib or vehicle as described under intravital microscopy. After 30 min, anesthetized female mice were injected intradermally with rabbit IgG anti-bovine serum albumin Abs (60 μg/30 μl; Sigma), followed immediately thereafter by an i.v. injection of BSA (500 μg/mouse; Sigma) (see Utomo et al., J Immunol, 180: 6279-6287, 2008). The intradermal injection of PBS served as a negative control. In cases where edema was measured, the solution of BSA contained 0.15% Evans blue dye (Sigma). The skin was harvested 3 h later.

Edema was evaluated by measuring the vascular leak of Evans Blue. Harvested skin was incubated with 1 ml DMF and Evans blue in the supernatant was quantified by measuring the absorbance at 650 nm. Specific edema formation was measured by subtracting the absorbance in the PBS-injected site from that of the IgG-challenged site in the same mouse.

For tissue neutrophil accumulation, an MPO assay was performed. MPO activity in supernatants of homogenized kidney tissue was measured with a TMB substrate kit (Pierce). Total protein content was measured with a BCA Protein Assay Kit (Pierce) and the quantity of MPO was normalized to protein content.

Nephritis Models

Nephrotoxic Serum Nephritis

Experimental nephrotoxic serum (NTS) nephritis was induced in FcγRIIA+/γ^{-/-} mice as previously reported²⁵. In brief, mice were preimmunized subcutaneously in the right footpad with 0.05 mg rabbit IgG (Jackson ImmunoResearch Laboratories) in Freund incomplete adjuvant and nonviable desiccated Mycobacterium tuberculosis H37Ra (Difco, Mich.). The mice were injected intravenously with 50 μl heat-inactivated, filter-sterilized rabbit NTS and given an oral administration of Bosutinib (150 mg/kg) or a vehicle solution every 24 hrs from day 0 to 21. The kidneys were harvested at day 21. Spot urine samples and peripheral blood were collected at indicated time points after NTS injection. Both kidneys from euthanized mice were harvested for histological analysis at day 14 or 21.

Human Lupus Serum Induced Kidney Injury Model

Human serum samples were obtained from the Lupus Clinic at the Beth Israel Deaconess Medical Center from patients with SLE diagnosed according to the classification criteria of the American College of Rheumatology as previously described¹³. Informed consent was obtained from all patients and healthy donors under a Beth Israel Deaconess Medical Center Institutional Review Board-approved protocol. FcγRIIA+/γ^{-/-}/Mac1-mice were injected s.c. in both flanks with 2.5 μg human IgG (Jackson ImmunoResearch Laboratories) in CFA (Thermo Scientific and Difco) on day-3. On days 0 and 2, 200 μl sterile serum was injected i.v. The mice were given an oral administration of Bosutinib (150 mg/kg) or a vehicle solution every 24 hrs from day 0 to 14. Spot urine samples and peripheral blood were collected at indicated time points after human serum injection. Both kidneys from euthanized mice were harvested for histological analysis at day 10 or 14.

Urine Analysis

Spot urine samples were collected and urine albumin and creatinine were evaluated by ELISA (Bethyl Laboratories) and expressed as a ratio of urine albumin to creatinine as previously reported²⁵.

Neutrophil Gelatinase Associated Lipocalin Analysis.

Urine from SLE sera treated mice were examined for NGAL protein levels using the manufacturer's protocol (ENZO Life Science).

Clinical Scoring of Arthritis

Mice were evaluated every other day after induction of disease and inflammation of each limb was scored, as previously reported (Baharav et al., J Rheumatol, 32: 469-476, 2005): 0, no evident inflammation; 1, redness or swelling of 1 toe; 2, redness or swelling of >1 toe; 3, ankle or tarsal-metatarsal involvement; 4, redness or swelling of the entire paw.

Histological Analysis and Scoring of Renal Tissue

Kidneys were fixed in formalin and paraffin embedded, or frozen in OCT medium, and 5-μm sections were prepared. For kidneys, periodic acid-Schiff, H&E, and dichloroacetate esterase (to identify neutrophils) on paraffin-embedded sections were performed, as described (Tang et al., J Exp Med, 186: 1853-1863, 1997). Neutrophils in 100 glomerular cross-sections were quantified and presented as neutrophils per glomerular cross-section. Immunohistochemistry on frozen sections was performed using a two-layer peroxidase method.

Tissue sections were blindly evaluated. For the NTS model in FcγRIIA+/γ^{-/-} mice, glomerular crescent formation and tubulointerstial injury was scored as previously described¹². For human lupus induced kidney injury in FcγRIIA+/γ^{-/-}/Mac1-mice, the histological score included endocapillary proliferation, leukocyte infiltration, and crescents, as previously described¹³. For joint lesions, histological scores reflected leukocyte infiltration, synovial thickening, and cartilage and bone erosion.

Two Photon Intravital Microscopy of the Glomerulus

10⁷ isolated FcγRIIA⁺/γ^{-/-} mouse bone marrow neutrophils were stained with either Cell Tracker Green CMFDA (5 μM, Life Technologies) or Cell Tracker Blue CMF2HC (5 μM, Life Technologies), washed and incubated with 1 uM Bosutinib or DMSO vehicle, respectively. FcγRIIA⁺/γ^{-/-} mice were injected intravenously with anti-GBM sera. 1.5 hrs later the labelled neutrophils were injected intravenously and 30 min thereafter the mice were prepared for intravital microscopy to image glomeruli. The dwell time of green or blue labeled at a certain locus were measured by tracking single leukocytes over several frames. Three glomeruli per mouse were analyzed.

Statistical Analysis

All data obtained are presented as the mean±SEM. Statistical differences were analyzed with the unpaired t-test. P values less than 0.05 were considered significant.

### EXAMPLE 1

### Bosutinib, Identified in a High Throughput Chemical Screen, is a Potent Inhibitor of FcgRIIA Activation In Vitro

An automated high-throughput drug screen in primary neutrophils from mice expressing FcγRIIA on neutrophils and lacking all their endogenous activating murine FcγRs (FcγRIIA⁺/γ^{-/-}) was established to identify compounds that inhibit the activity of FcγRIIA. Immune complex (IC)-induced reactive oxygen species (ROS) generation served as our read-out of FcγRIIA activity. An appropriate positive (PP2, an inhibitor of the FcγR proximal Src-family kinase) and negative (DMSO, vehicle) control was included in the assay. The screen of 8,483 compounds, used at 10 μM in DMSO, yielded 84 hits (1% hit rate) that were defined as those that have an inhibitory potential that is ≧95% of PP2 (FIG. 1A). Of the 30 compounds cherry picked for a secondary screen, ten compounds were identified as having an ability to inhibit neutrophil FcyRIIA induced ROS generation at even lower concentrations (i.e., 1.1 μM or less) (Table 1).

TABLE 1
List of top 10 biologically active compounds that significantly
suppressed mouse BMN reactive oxygen species (ROS) generation
induced by immobilized IC.

| Compounds | | ROS |
|---|---|---|
| (1.1 μM) | Mode of action | RLU ratio |
| Bosutinib | Bcr-Abl and Src-Abl kinase inhibitor | 0.033 |
| A-443654 | Akt inhibitor | 0.266 |
| PF431396 | FAK and PYK2 inhibitor | 0.301 |
| GF109203X | PKC inhibitor | 0.338 |
| Ro 31-8220 | PKC inhibitor | 0.778 |

Among these, Bosutinib exhibited the most potent activity (FIG. 1B) with an IC50 of approximately 50 nM (FIG. 1C). Bostuinib also effectively reduced ROS generation in a dose dependent manner following direct cross-linking of FcyRIIA with antibody, which represents another method for inducing ROS (FIG. 1D). Next, cells were challenged with other stimuli known to induce a respiratory burst to assess the specificity of Bosutinib. The fungal component zymosan engages Dectin-1, which like FcγR uses its immunoreceptor tyrosine-activating motif (ITAM) to induce ROS production²¹⁻²². Bosutinib inhibited ROS generation induced by zymosan but to a milder degree compared to FcγR cross-linking It had no effect even at high doses, on ROS generation following stimulation with phorbol myristate acetate (PMA), a potent inducer of ROS generation through direct activation of Protein kinase C (FIG. 1E). The latter result suggests that Bosutinib does not target a final common pathway of ROS generation and is not cytotoxic to cells. Consistent with this, Bosutinib, at a range of concentrations did not affect cell viability as assessed by Annexin V and propium iodide.

Bosutinib also inhibited other FcγRIIA mediated functions such as immune complex induced release of the neutrophil cytotoxic granule components myeloperoxidase and elastase (FIG. 1F) and some effect on neutrophil spreading on immobilized ICs (FIG. 1G). As observed for mouse neutrophils, Bosutinib potently inhibited ROS generation (FIG. 1H) and degranulation (FIG. 1I) in human peripheral blood neutrophils in a dose dependent manner. Activation of FcγRIIA is initiated by the phosphorylation of its ITAM motif, which triggers the assembly of protein complexes containing Syk- and Src-family tyrosine kinases. This results in the subsequent activation of a number of downstream signaling molecules include the Rho GTPase guanine exchange factor (GEF) Vav, PI3K, the non-receptor tyrosine kinase Pyk-2 as well as the NADPH oxidase component p40 phox. Western blot analysis of human neutrophils revealed that Bosutinib inhibited FcγRIIA mediated phosphorylation of Vav, Akt (a target of PI3K), Pyk2, p40 phox , p-Crkl (a read-out of Abl activity) and ERK (a MAPK) (FIG. 1J). Similar results were obtained for mouse neutrophils harvested from FcγRIIA/γ^{-/-} mice (FIG. 1K). The reduction in FcγR dependent activation of Vav and Pyk2 observed in Bosutinib treated FcγRIIA/γ^{-/-} neutrophils was similar to that in neutrophils deficient in all three Src family kinases (FIG. 1K). The similar reduction in indicated proteins following Bosutinib treatment or Src deficiency suggests the possibility that some of the effects of Bosutinib on FcγRIIA signaling may be the result of Bosutinib's reported effects on Src kinases³³.

### EXAMPLE 2

### Bosutinib Inhibits FcγRIIA Dependent Neutrophil Recruitment and Tissue Injury

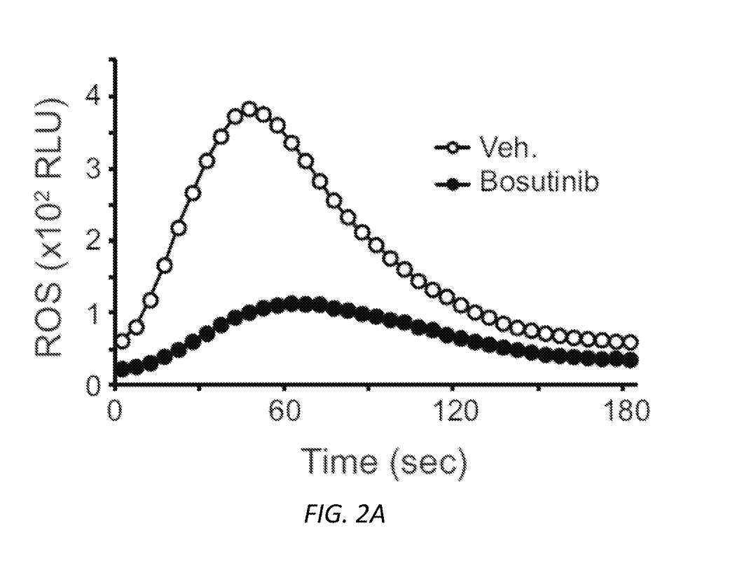

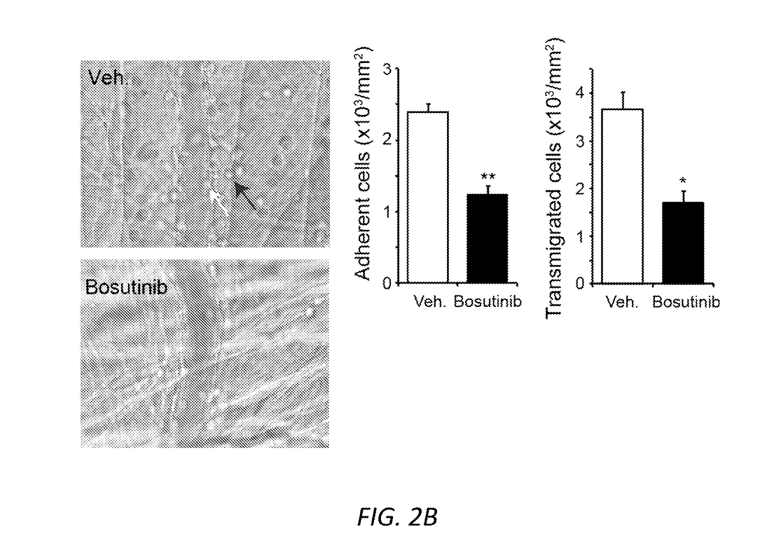

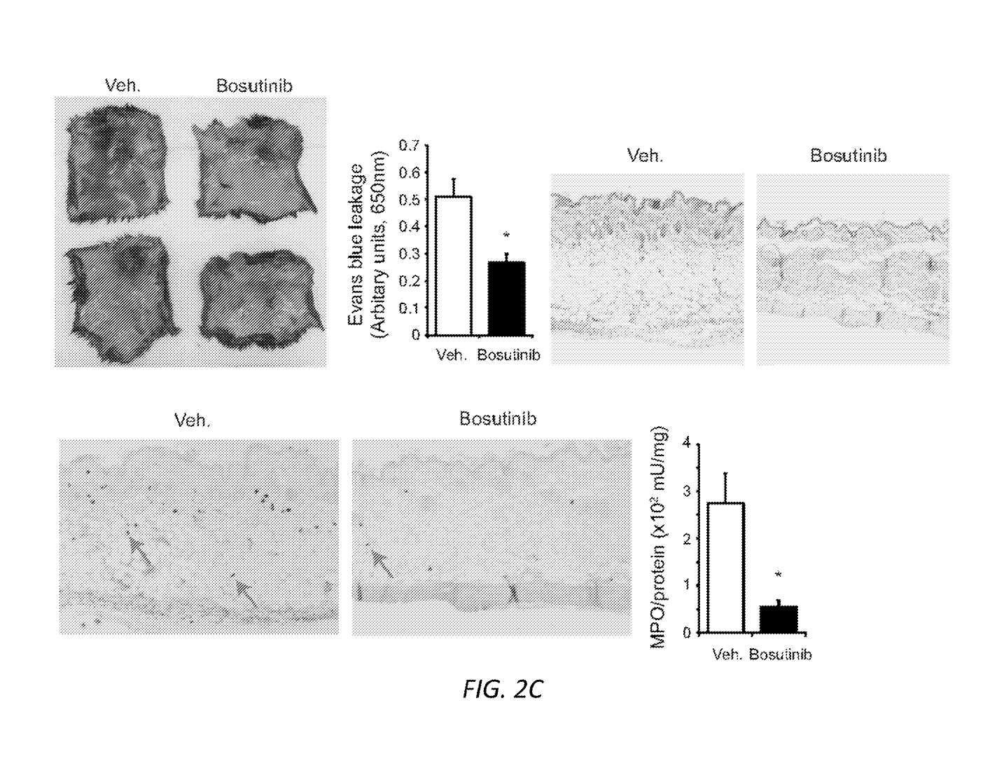

Bosutinib is an orally administered medication¹⁵. When given by gavage to mice, Bosutinib's blood concentration peaks at 2-4 hours²³. Peripheral blood neutrophils of mice analyzed 4 hrs after a gavage with 150 mg/kg Bosutinib exhibited a significant reduction in their ability to generate ROS ex vivo following FcγRIIA cross-linking (FIG. 2A), suggesting effective doses of Bosutinib in vivo. The effect of a single administration of Bosutinib on the RPA reaction in the cremaster and skin were assessed. The RPA reaction results in FcγRIIA dependent neutrophil recruitment and edema (i.e., plasma leakage)¹² upon in situ IC formation induced by the local administration of antibody and the intravenous delivery of antigen. Intravital microscopy of the cremaster subjected to the RPA reaction revealed that Bosutinib significantly attenuated neutrophil adhesion and transmigration (FIG. 2B). Furthermore, in the cutaneous RPA reaction, Bosutinib reduced edema and neutrophil recruitment (FIG. 2C).

### EXAMPLE 3

### Bosutinib Inhibits FcγRIIA Dependent Renal Injury in Nephrotoxic Nephritis and in Lupus Nephritis Induced by Human SLE Serum

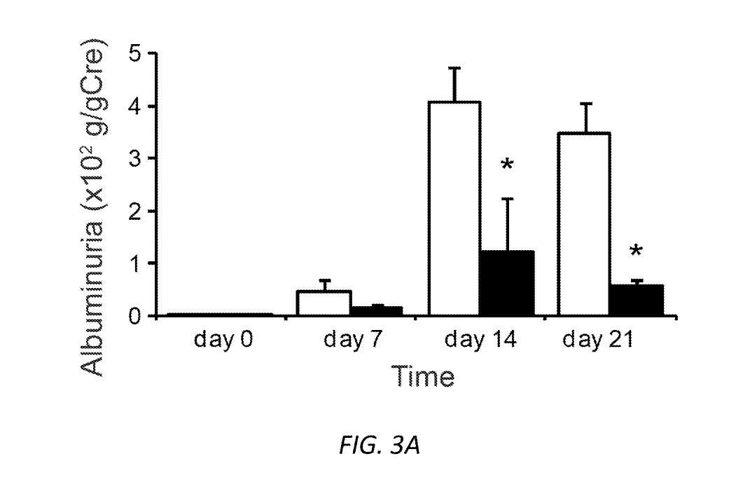

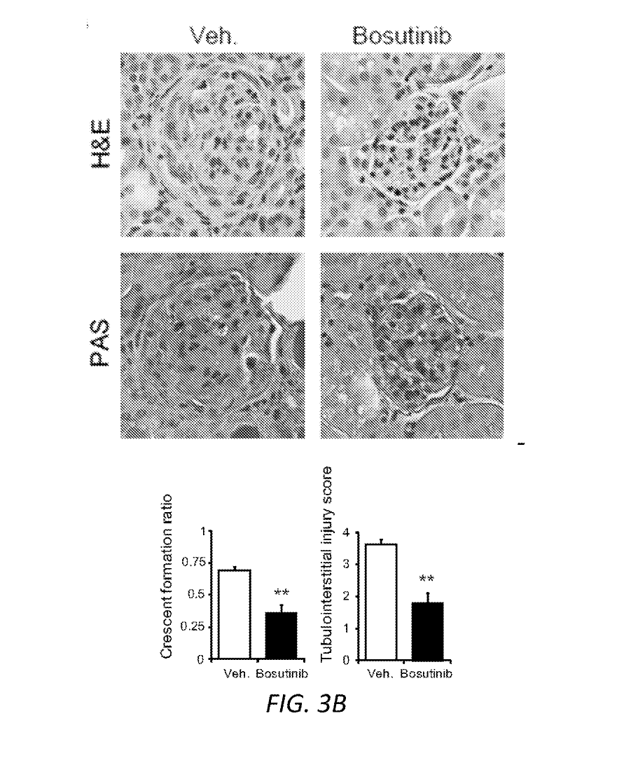

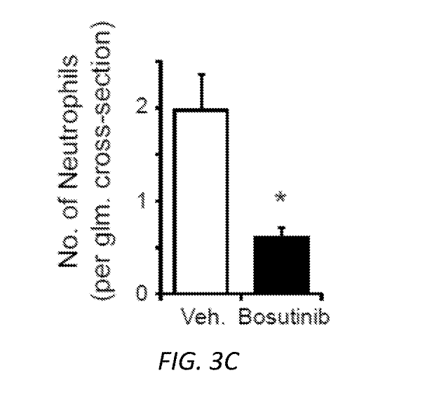

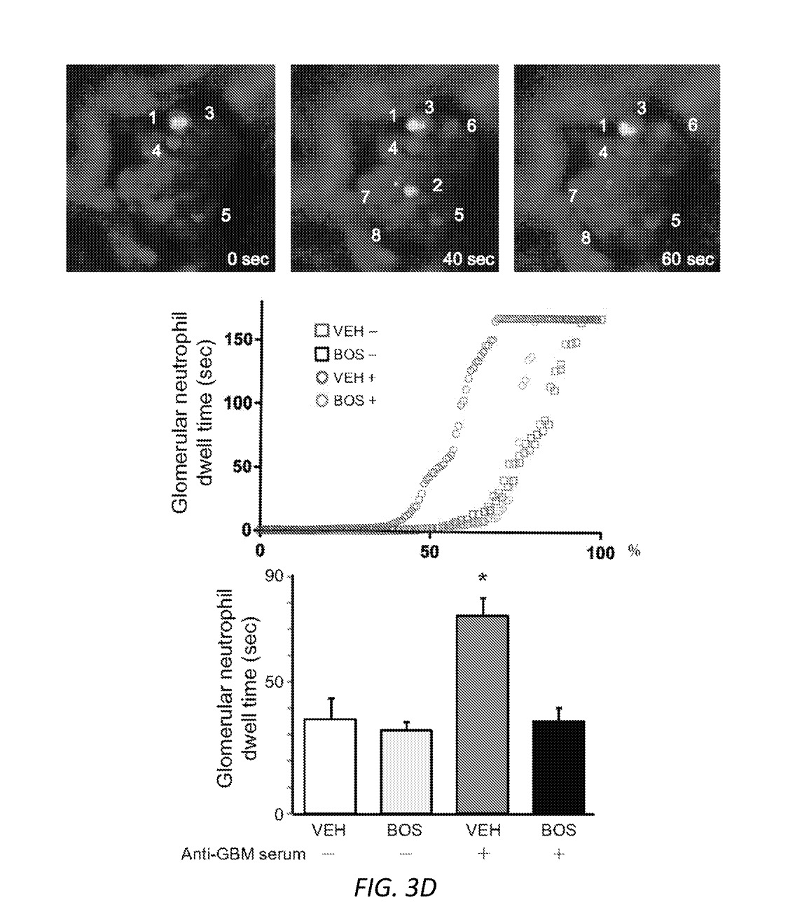

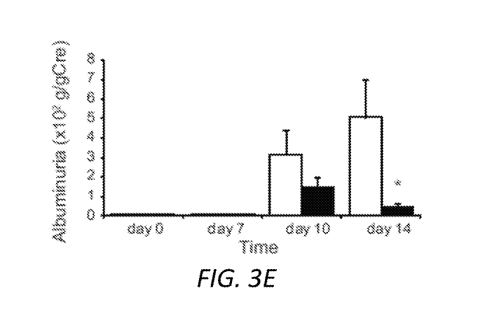

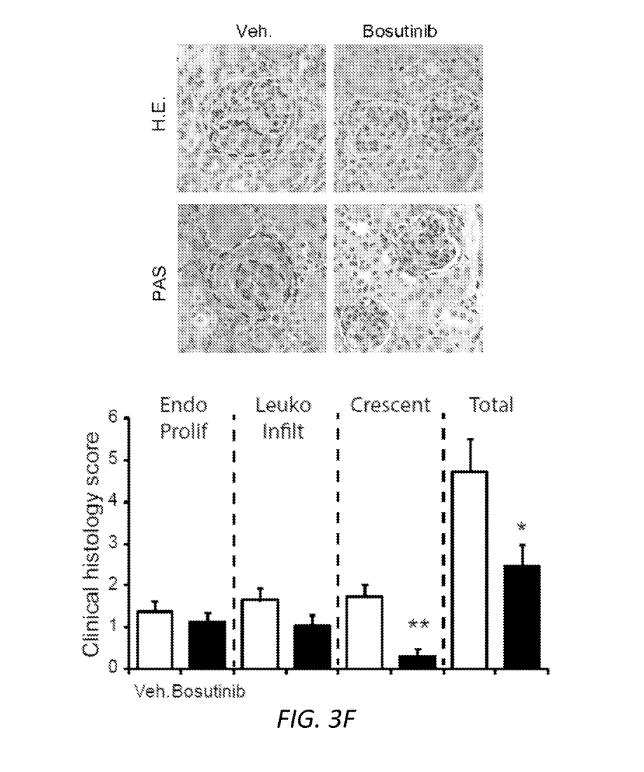

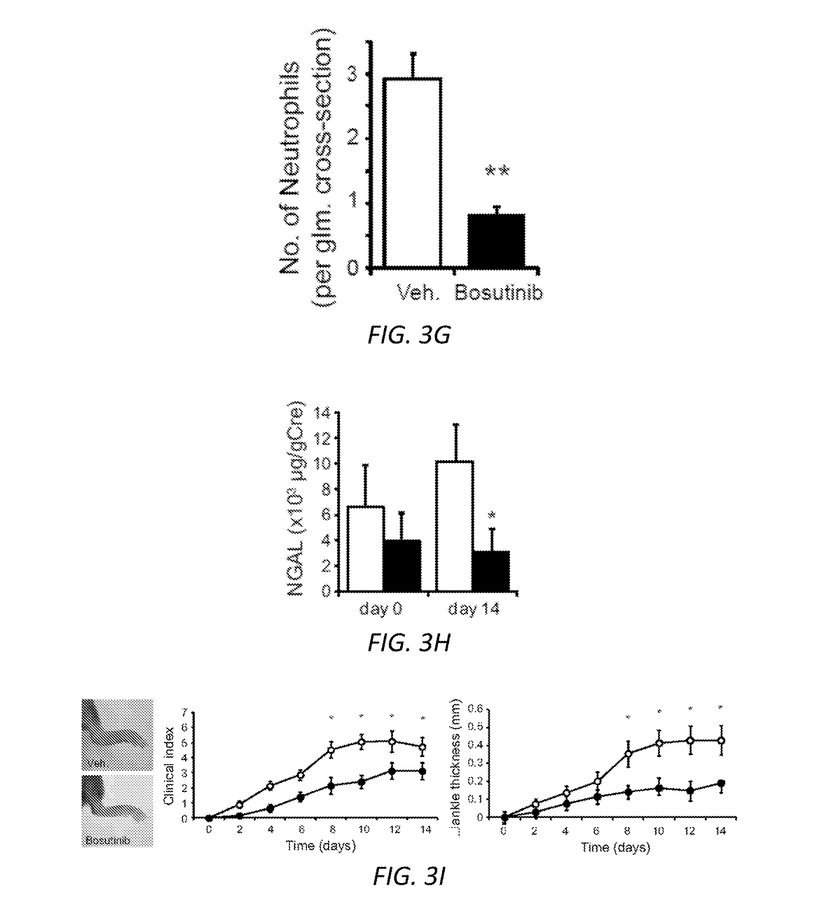

Nephrotoxic serum nephritis²⁴, a model of Goodpasture disease³⁰⁻³¹ and a potential surrogate for lupus nephritis¹⁶, is a FcγRIIA mediated disease model in mice¹² that is characterized by significant protein leakage in the urine, a hallmark of glomerular injury. This correlates with renal histological changes including glomerular hypercellularity, crescent formation and tubular dilation and atrophy, which develops over 14-21 days²⁵. Daily oral administration of Bosutinib versus vehicle alone markedly reduced albuminuria in FcγRIIA mice subjected to nephrotoxic nephritis (FIG. 3A). Histological analysis of kidney sections demonstrated that glomerular crescent formation and tubulointerstitial injury (FIG. 3B) as well as neutrophil infiltration (FIG. 3C) were significantly attenuated by Bosutinib treatment.

To directly examine the effect of Bosutinib on neutrophils in altering glomerular neutrophil accumulation following anti-GBM antibody deposition in the kidney, neutrophils were isolated from FcγRIIA/γ^{-/-} mice, divided in two groups, labeled with two different fluorophores and treated with vehicle control or Bostutinib, and intravenously injected into mice that were given anti-GBM serum (+) or saline (−). Behavior of the neutrophils in the two groups were simultaneously imaged in the glomeruli of the kidney in real time by 2-photon intravital microscopy following exteriorization of the kidney of anesthetized mice (FIG. 3D). In mice given anti-GBM, accumulation of Bosutinib treated neutrophils was significantly reduced compared to vehicle treated counterparts as assessed by a reduction in dwell time of the neutrophils within glomerular capillaries. Indeed, the accumulation of Bosutinib treated neutrophils in anti-GBM treated recipient mice was reduced to that observed in saline treated (i.e., no anti-GBM) recipients (FIG. 3D).

Mice expressing FcyRIIA and lacking Mac-1 are susceptible to developing lupus nephritis and arthritis with 14 days of the passive transfer of human lupus sera¹³. This model has relevance for human disease as a Mac-1 SNP, R77H, significantly associates with lupus risk and results in a reduction in Mac-1 function (data not shown). As previously described, mice were preimmunized with human IgG/CFA on day-3 and given two intravenous injections of SLE sera at days 0 and 2¹³. Bosutinib or vehicle control was given by gavage daily starting at day 0. SLE serum caused marked proteinuria by day 10 and 14 in the vehicle treated group. In contrast, mice given Bosutinib daily starting at day 0, exhibited a marked reduction in albuminuria at day 10 and 14 (FIG. 3E). Bosutinib markedly reduced crescent formation, a histological hallmark of severe glomerular injury (FIG. 3F), glomerular neutrophil accumulation (FIG. 3G). It also reduced the accumulation of a neutrophil derived product, Neutrophil Gelatinase Associated Lipocalin (NGAL) in the urine of mice (FIG. 3H), suggesting the possibility that NGAL may serve as a surrogate indicator of the effectiveness of Bosutinib in vivo. A subset of animals treated with vehicle control also developed arthritis. The incidence of arthritis manifested as local redness and swelling of four limb joints and ankle joint thickness was significantly reduced in the Bosutinib treated group (FIG. 3I). Bosutinib can cause leukopenia in some clinical settings³². Hemocytometric analysis showed comparable total leukocyte and neutrophil counts in vehicle- and Bosutinib-treated mice at day 14 (Table 2).

TABLE 2
Peripheral blood cell counts in the mouse model with
human lupus serum induced glomerular injury at day 14

| | | WBC | Neu | | RBC | Hb | Plt |
|---|---|---|---|---|---|---|---|
| | | total, K/ul | total, K/ul | % | M/ul | g/dl | total, K/ul |
| AVE | Vehicle | 2.294286 | 0.415575 | 17.77714 | 9.014286 | 9.014286 | 1053.714 |
| | Bosutinib | 2.656 | 0.750569 | 25.64 | 7.98 | 7.98 | 949.8 |
| SEM | Vehicle | 0.330417 | 0.071319 | 1.661645 | 0.809888 | 0.809888 | 77.66494 |
| | Bosutinib | 0.575027 | 0.25364 | 3.671587 | 0.307246 | 0.307246 | 88.13932 |
| P value | | 0.603411 | 0.263595 | 0.101913 | 0.26833 | 0.26833 | 0.399395 |

WBC, white blood cells;
Neu, neutrophils;
RBC, red blood cells;
Hb, hemoglobin;
Plt, platelets

### EXAMPLE 4

### Silencing of c-Abl Tyrosine Kinase in a Human Myeloid Cell Line Inhibits FcγRIIA Mediated ROS Generation

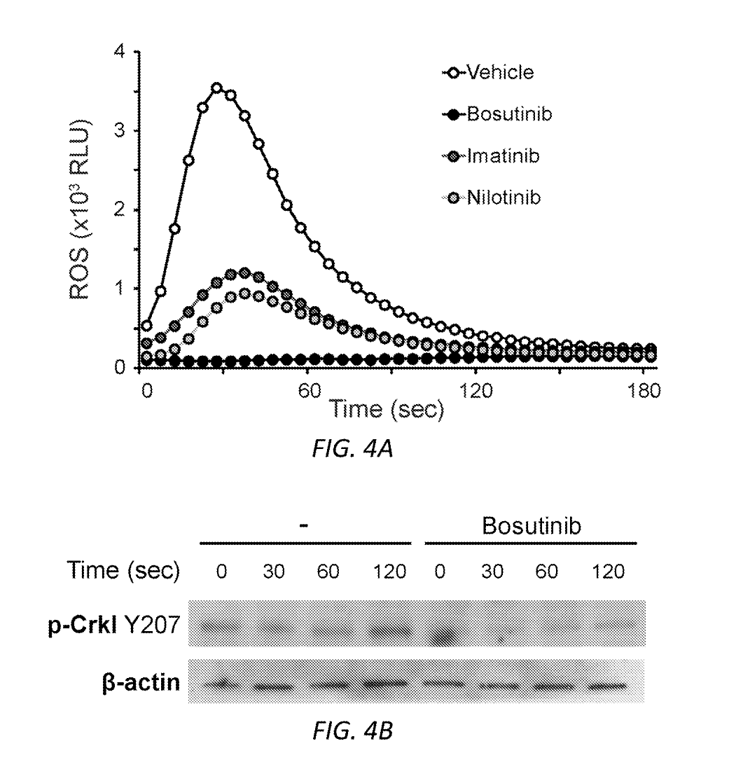

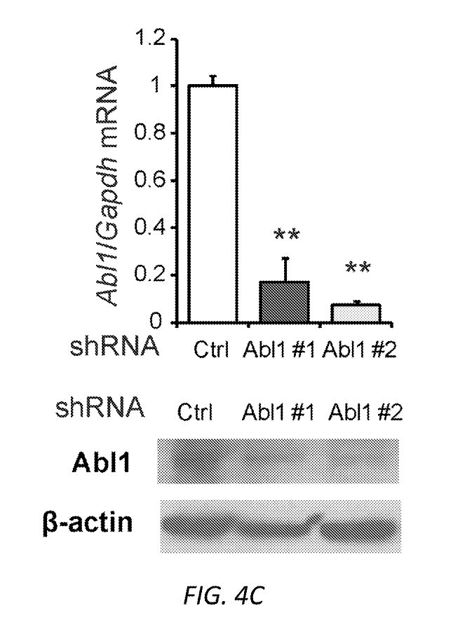

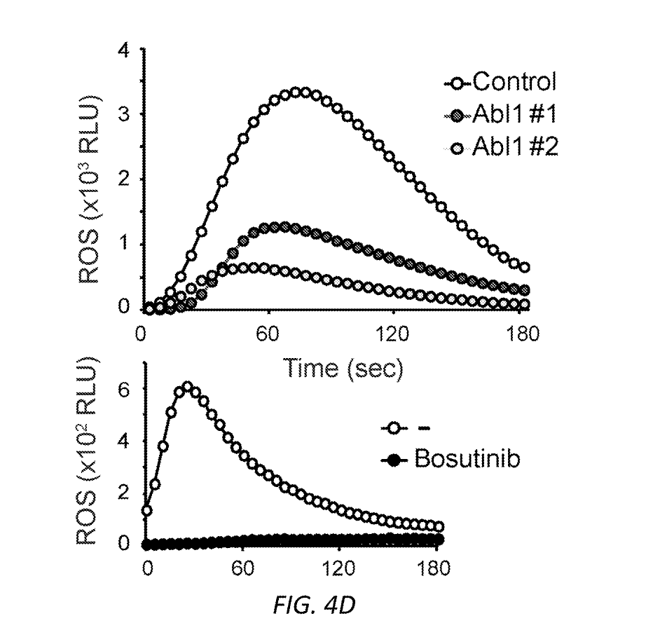

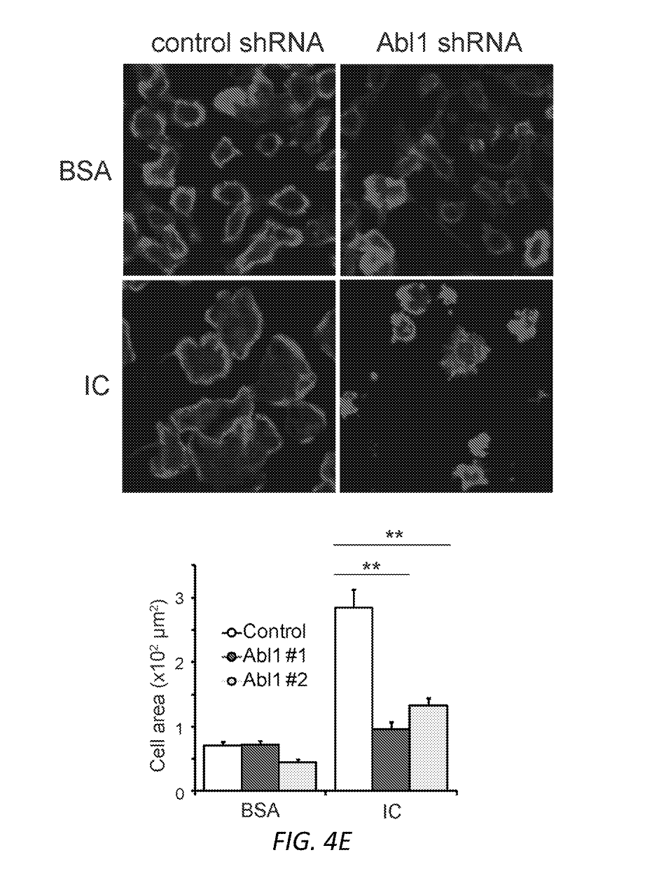

Bosutinib is an inhibitor of Bcr-Abl, a fusion protein of Bcr (whose normal function remains unclear) and the non-receptor tyrosine kinase Abl²⁷, that results from a chromosomal abnormality in more than 90% of CML patients and is also described as an inhibitor of Src kinase³³. Currently multiple generations of Bcr-Abl tyrosine-kinase inhibitors are available such as Imatinib, Nilotinib, and Bosutinib. Both Imatinib and Nilotinib reduced neutrophil ROS generation induced by FcyRIIA cross linking although the potency of two compounds was lower compared to Bosutinib (FIG. 4A). The higher potency of Bosutinib may be due to its ability to also inhibit Src kinases. Next, we determined whether Abl1 is required for neutrophil FcyRIIA signaling and that it is a target of Bosutinib in the context of FcyRIIA mediated cytotoxicity. Biochemical analysis showed that FcγRIIA cross linking in mouse neutrophils induced the phosphorylation of Crkl, a downstream target of Bcr/Abl1²⁸, and that the phosphorylation was inhibited by Bosutinib (FIG. 4B). Next, the functional role of Abl1 in FcγRIIA signaling was assessed. Differentiated human neutrophil like HL-60 cells expressing FcγRIIA were generated with stable suppression of Abl1 mRNA using lentiviral vectors expressing shRNAs against Abl1 sequences (FIG. 4C). Silencing of Abl1 using two different shRNA sequences resulted in a significant reduction in ROS generation induced by FcγRIIA activation (FIG. 4D), as did Bosutinib treatment of Abl1 expressing HL60 cells (FIG. 4D). The significant reduction in ROS with Abl1 shRNA alone suggests a non-redundant role for Abl1 versus Abl2 (Abelson related gene, Arg)²⁹ in FcγRIIA mediated ROS generation. Cell spreading on IC-coated plate was also reduced by Abl1 silencing (FIG. 4E). These findings indicate that Abl1 is required to efficiently activate FcγRIIA in neutrophils and infer that targeting of this kinase is significantly responsible for Bosutinib's effects.

### REFERENCES

1. Mayadas et al., Mechanisms of immune complex-mediated neutrophil recruitment and tissue injury. Circulation, 120: 2012-2024, 2009.

2. Nimmerjahn and Ravetch, Fcgamma receptors as regulators of immune responses. Nat Rev Immunol, 8: 34-47, 2008.

3. Ivashkiv, Cross-regulation of signaling by ITAM-associated receptors. Nat Immunol, 10: 340-347, 2009.

4. Dunn-Siegrist et al., Pivotal involvement of Fcgamma receptor HA in the neutralization of lipopolysaccharide signaling via a potent novel anti-TLR4 monoclonal antibody 15C1. J Biol Chem, 282: 34817-34827, 2007.

5. Duits et al., Skewed distribution of IgG Fc receptor IIa (CD32) polymorphism is associated with renal disease in systemic lupus erythematosus patients. Arthritis Rheum, 38: 1832-1836, 1995.

6. Villarreal et al., Mannose binding lectin and FcgammaRIIa (CD32) polymorphism in Spanish systemic lupus erythematosus patients. Rheumatology (Oxford), 40: 1009-1012, 2001.

7. Radstake et al., Role of Fcgamma receptors IIA, IIIA, and IIIB in susceptibility to rheumatoid arthritis. J Rheumatol, 30: 926-933, 2003.

8. Jostins et al., Host-microbe interactions have shaped the genetic architecture of inflammatory bowel disease. Nature, 491: 119-124, 2012.

9. Alizadeh et al., Association analysis of functional variants of the FcgRIIa and FcgRIIIa genes with type 1 diabetes, celiac disease and rheumatoid arthritis. Hum Mol Genet, 16: 2552-2559, 2007.

10. Smith et al., Mouse model recapitulating human Fcgamma receptor structural and functional diversity. Proc Natl Acad Sci U S A, 109: 6181-6186, 2012.

11. Tan Sardjono et al., Development of spontaneous multisystem autoimmune disease and hypersensitivity to antibody-induced inflammation in Fcgamma receptor IIa-transgenic mice. Arthritis Rheum, 52: 3220-3229, 2005.

12. Tsuboi et al., Human neutrophil Fcgamma receptors initiate and play specialized nonredundant roles in antibody-mediated inflammatory diseases. Immunity, 28: 833-846, 2008.

13. Rosetti et al., Human lupus serum induces neutrophil-mediated organ damage in mice that is enabled by Mac-1 deficiency. J Immunol, 189: 3714-3723, 2012.

14. Tsuboi et al., Regulation of human neutrophil Fcgamma receptor IIa by C5a receptor promotes inflammatory arthritis in mice. Arthritis Rheum, 63: 467-478, 2011.

15. Golas et al., SKI-606, a 4-anilino-3-quinolinecarbonitrile dual inhibitor of Src and Abl kinases, is a potent antiproliferative agent against chronic myelogenous leukemia cells in culture and causes regression of K562 xenografts in nude mice. Cancer Res, 63: 375-381, 2003.

16. Du et al., Experimental anti-GBM nephritis as an analytical tool for studying spontaneous lupus nephritis. Arch Immunol Ther Exp (Warsz), 56: 31-40, 2008.

17. Sthoeger et al., Novel approaches to the development of targeted therapeutic agents for systemic lupus erythematosus. J Autoimmun, 2014.

18. Rovin and Parikh, Lupus nephritis: the evolving role of novel therapeutics. Am J Kidney Dis, 63: 677-690, 2014.

19. Borchers et al., Lupus nephritis: a critical review. Autoimmun Rev, 12: 174-194, 2012.

20. Shen et al., Bosutinib in the treatment of patients with Philadelphia chromosome-positive (Ph+) chronic myelogenous leukemia: an overview. Ther Adv Hematol, 5: 13-17, 2014.

21. Taylor et al., Dectin-1 is required for beta-glucan recognition and control of fungal infection. Nat Immunol, 8: 31-38, 2007.

22. Boyle et al., Class IA phosphoinositide 3-kinase beta and delta regulate neutrophil oxidase activation in response to Aspergillus fumigatus hyphae. J Immunol, 186: 2978-2989, 2011.

23. Golas, et al., SKI-606, a Src/Abl inhibitor with in vivo activity in colon tumor xenograft models. Cancer Res, 65: 5358-5364, 2005.

24. Masugi, Uber die experimentelle glomerulonephritis durch das spezifische Antinierenserum. Ein Beitrag zur Pathogenese der diffusen glomerulonephritis. Beitr Pathol Anat Allg Pathol, 92: 17, 1934.

25. Rosenkranz, et al., P-selectin deficiency exacerbates experimental glomerulonephritis: a protective role for endothelial P-selectin in inflammation. J Clin Invest, 103: 649-659, 1999.

26. Cortes et al., Bosutinib versus imatinib in newly diagnosed chronic-phase chronic myeloid leukemia: results from the BELA trial. J Clin Oncol, 30: 3486-3492, 2012.

27. Wang, The capable ABL: what is its biological function? Mol Cell Biol, 34: 1188-1197, 2014.

28. de Jong et al., Tyrosine 207 in CRKL is the BCR/ABL phosphorylation site. Oncogene, 14: 507-513, 1997.

29. Wong and Witte, The BCR-ABL story: bench to bedside and back. Annu Rev Immunol, 22: 247-306, 2004.

30. Pedchenko, V, Vanacore, R, Hudson, B: Goodpasture's disease: molecular architecture of the autoantigen provides clues to etiology and pathogenesis. Curr Opin Nephrol Hypertens, 20: 290-296, 2011.

31. Pusey, CD: Anti-glomerular basement membrane disease. Kidney Int, 64: 1535-1550, 2003.

32. Cortes, J: Bosutinib in the treatment of chronic myelogenous leukemia. Clin Adv Hematol Oncol, 10: 736-737, 2012.

33. Boschelli, D H, Ye, F, Wang, Y D, Dutia, M, Johnson, S L, Wu, B, Miller, K, Powell, D W, Yaczko, D, Young, M, Tischler, M, Arndt, K, Discafani, C, Etienne, C, Gibbons, J, Grod, J, Lucas, J, Weber, J M, Boschelli, F: Optimization of 4-phenylamino-3-quinolinecarbonitriles as potent inhibitors of Src kinase activity. J Med Chem, 44: 3965-3977, 2001.

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A method of treating a condition associated with IgG-immune complex-mediated organ damage in a subject, the method comprising administering a therapeutically effective amount of a Bcr-Abl tyrosine-kinase inhibitor to the subject.

2. The method of claim 1, wherein the condition is lupus.

3. The method of claim 5, wherein the condition is lupus nephritis.

4. The method of claim 1, wherein the condition is Goodpasture syndrome.

5. The method of claim 1, wherein the Bcr-Abl tyrosine-kinase inhibitor is Imatinib, Nilotinib, or Bosutinib.

6. The method of claim 5, wherein the Bcr-Abl tyrosine-kinase inhibitor is Bosutinib.

7.-12. (canceled)

13. The method of claim 1, wherein the organ damage is kidney, liver, pancreas, lung, heart/cardiovascular system, or joint damage.

14. The method of claim 13, wherein the organ damage is kidney damage.

15. The method of claim 1, wherein the subject does not have Chronic myelogenous leukemia (CML).

16. The method of claim 1, further comprising selecting a subject for treatment on the basis that they have lupus or Goodpasture syndrome.
